(19) United States
(12) Patent Application Publication
(10) Pub. No.: US 2026/0123894 A1
(43) Pub. Date: May 7, 2026

(54) Multimodal Predicate Diagnostic System for Hospital Stroke Detection

(71) Applicant: George William Bickerstaff, III, Greenwich, CT (US)

(72) Inventor: George William Bickerstaff, III, Greenwich, CT (US)

(21) Appl. No.: 19/440,285

(22) Filed: Jan. 5, 2026

(51) Int. Cl. A61B 5/00; A61B 5/11; G16H 50/30

(52) U.S. Cl. CPC A61B 5/7275; A61B 5/112; A61B 5/7221; A61B 5/7264; A61B 5/746; G16H 50/30

(57) ABSTRACT

A multimodal predicate diagnostic system for hospital stroke detection utilizes TEE-secured mobile devices to monitor patients in clinical environments. The system aggregates motion data using optimized sampling and cryptographic validation, applying a hybrid CNN-decision tree model optimized via transfer learning and data augmentation against an adaptive gait baseline. It computes risk confidence scores in real-time and generates alerts with hospital network integration.

### CROSS-REFERENCE TO RELATED APPLICATIONS

None.

### STATEMENT REGARDING FEDERALLY SPONSORED RESEARCH OR DEVELOPMENT

Not applicable.

### INCORPORATION BY REFERENCE

None.

### FIELD OF THE INVENTION

The present invention relates to mobile health monitoring and cryptographic diagnostic systems within clinical environments. Specifically, it relates to a computer-implemented predicate diagnostic framework that analyzes multimodal ambulatory data from Trusted Execution Environment (TEE)-secured devices using adaptive gait baselines for hospital-based stroke and TIA detection.

### BACKGROUND OF THE INVENTION

Stroke and transient ischemic attacks (TIAs) require constant, high-precision monitoring in hospital wards to prevent secondary events and ensure rapid response. Traditional diagnostics often rely on intermittent nurse observations or stationary equipment. Existing mobile solutions lack the integrated cryptographic security and real-time clinical system connectivity required to safely monitor ambulatory patients within a high-density hospital network environment.

### SUMMARY OF THE INVENTION

The invention provides a predicate diagnostic system for detecting stroke and TIAs in hospital settings using TEE-secured devices. The system comprises a sensor data aggregator optimized for clinical hardware, a predicate analysis engine with an adaptive gait baseline, a stroke risk classifier, and an alert module. By utilizing hospital connectivity protocols, hardware-level encryption, and an AI architecture optimized via transfer learning and data augmentation, the system identifies physiological deviations while maintaining strict HIPAA-compliant confidentiality.

### BRIEF DESCRIPTION OF THE DRAWINGS

FIG. 1: SYSTEM ARCHITECTURE
FIG 1A: SENSOR DATA AGGREGATOR CONFIGURATION—Illustrates the TEE-secured setup for collecting multimodal hospital data via clinical hardware. The configuration manages high-fidelity sensor inputs while balancing hospital network bandwidth. Hardware-level isolation is maintained throughout the aggregation process to protect patient privacy.
FIG. 1B: PREDICATE ANALYSIS ENGINE FLOW—Depicts the workflow for applying predicate rules using a hybrid CNN-decision tree model optimized for high-density hospital data. The engine processes multimodal inputs in real-time to identify acute clinical anomalies. Logic paths are detailed for differentiating between standard patient movement and acute stroke indicators.
FIG. 1C: STROKE RISK CLASSIFIER ALGORITHM—Shows the algorithm computing cryptographically validated risk scores in less than one second. The diagram highlights the interaction between the adaptive gait baseline and real-time sensor streams. Security protocols are embedded within the classifier to ensure risk data integrity.
FIG. 1D: ALERT MODULE INTEGRATION—Displays Integration With hospital local area networks utilizing less than two-second latency. The module manages the prioritized delivery of alerts to specific medical response teams. Network routing logic ensures notifications reach appropriate clinical terminals without interruption.
FIG. 1E: DEVICE SYNCHRONIZATION PROTOCOL—Describes the low-latency protocol for syncing hospital device data across multiple clinician terminals. The protocol maintains data continuity as patients move between clinical departments. Synchronization occurs within the secure environment to prevent interception on the local area network.

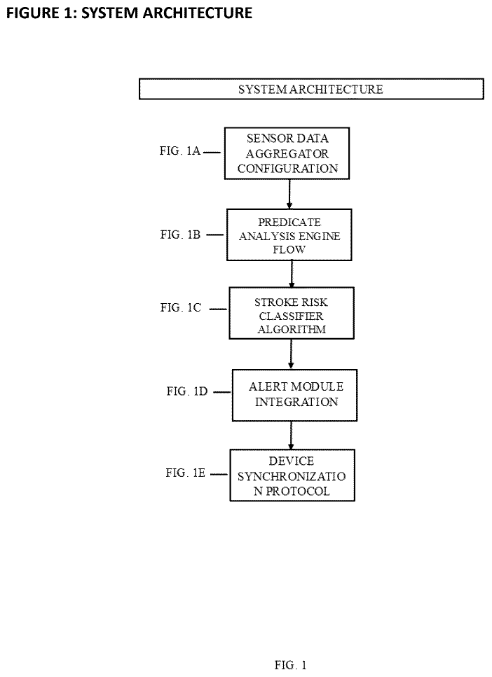

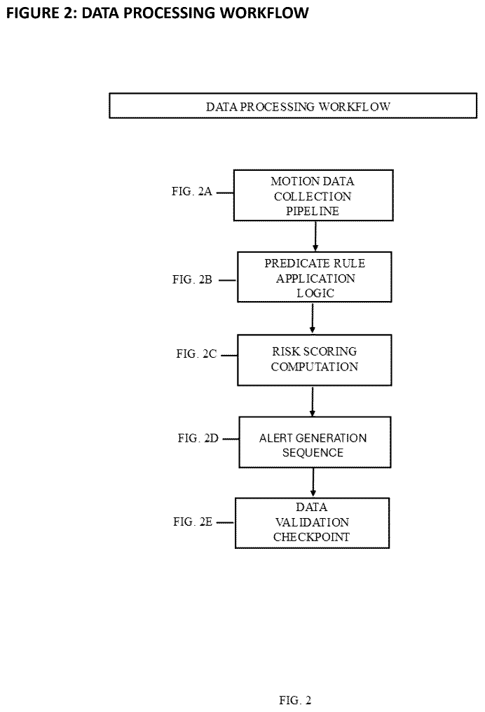

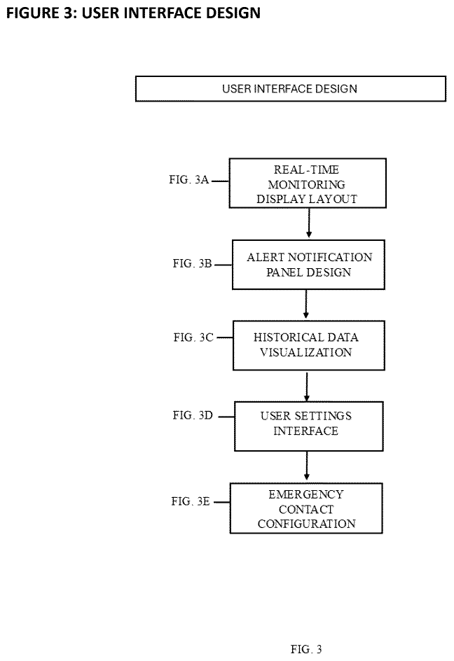

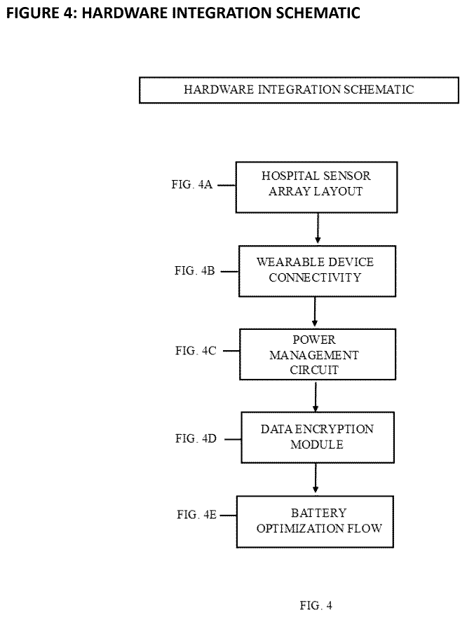

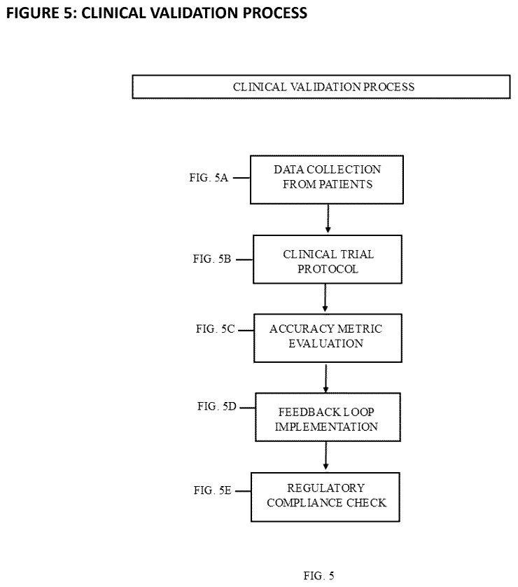

FIG. 2: DATA PROCESSING WORKFLOW
FIG. 2A: MOTION DATA COLLECTION PIPELINE—Outlines the pipeline for gathering multimodal hospital data with optimized sampling triggers. The pipeline includes specific stages for noise reduction and cryptographic signing. Real-time sensor fusion occurs here to prepare data for higher-level predicate analysis.
FIG. 2B: PREDICATE RULE APPLICATION LOGIC—Details the logic for evaluating stroke/TIA thresholds for patients in clinical settings. The logic incorporates environmental precision data to handle hospital-specific movement variables. The system provides actionable data to clinicians without making autonomous medical decisions.
FIG. 2C: RISK SCORING COMPUTATION—Illustrates the computation of cryptographically validated risk confidence scores. The calculation integrates patient-specific medical history with current physiological movement data. Results are formatted for immediate inclusion in digital medical records.
FIG. 2D: ALERT GENERATION SEQUENCE—Shows the Sequence of steps to generate and route alerts to medical teams. The sequence initiates immediately upon a risk score exceeding the predefined threshold. Automated logs are created to track the response time and clinical outcome of each alert.
FIG. 2E: DATA VALIDATION CHECKPOINT—Depicts Validation utilizing adaptive baselines and cryptographic signatures. This ensures movement data is correctly attributed to specific patients in crowded wards. Validation steps are performed within the TEE to maintain high-level security.

FIG. 3: USER INTERFACE DESIGN
FIG. 3A: REAL-TIME MONITORING DISPLAY LAYOUT—Presents the layout for hospital stroke monitoring with real-time waveform overlays. The layout allows clinicians to monitor multiple patients simultaneously from a central station. Status indicators provide immediate visual confirmation of system health and sensor connectivity.
FIG. 3B: ALERT NOTIFICATION PANEL DESIGN—Illustrates notification panel featuring patient bed location and stroke risk metrics. The panel uses color-coded priorities to guide medical staff during emergency events. Touch-sensitive elements allow clinicians to acknowledge alerts and view detailed sensor data.
FIG. 3C: HISTORICAL DATA VISUALIZATION—Shows trends recorded throughout the hospital stay for longitudinal analysis. Data is presented in a way that allows for easy comparison between recovery stages. Clinicians can zoom into specific events to review raw sensor waveforms for diagnostic verification.
FIG. 3D: USER SETTINGS INTERFACE—Depicts interface for configuring hospital-specific diagnostic modes. Settings allow for the adjustment of alert thresholds based on specific ward protocols. Access controls ensure only authorized medical personnel can modify system parameters.
FIG. 3E: EMERGENCY CONTACT CONFIGURATION—Describes setup for clinical coordination contacts utilizing cryptographic validation. The interface links patients to their assigned rapid response teams and primary neurologists. Configuration data is encrypted to comply with hospital privacy standards.

FIG. 4: HARDWARE INTEGRATION SCHEMATIC
FIG. 4A: HOSPITAL SENSOR ARRAY LAYOUT—Illustrates sensor layout with TEE security and clinical hardware integration. The schematic identifies the physical placement of accelerometers and clinical-grade motion sensors. Wiring and data paths are shown connecting to the centralized hospital diagnostic unit.
FIG. 4B: WEARABLE DEVICE CONNECTIVITY—Shows Low-latency protocol for clinical-grade wearables used in the ward. The diagram details the secure handshake between patient wearables and the bedside aggregator. Connection status is monitored continuously to prevent gaps in patient observation.
FIG. 4C: POWER MANAGEMENT CIRCUIT—Depicts Circuit Designed for continuous sampling in hospital environments. The circuit manages charging from clinical docks while maintaining monitoring operation. Fail-safe battery backups are included to handle hospital power fluctuations.
FIG. 4D: DATA ENCRYPTION MODULE—Illustrates Tee-based module utilizing AES-256 and SHA-3. The module creates a hardware-isolated environment for all cryptographic calculations. Secure keys are managed internally to prevent extraction by unauthorized software.
FIG. 4E: BATTERY OPTIMIZATION FLOW—Describes Flow for maximizing battery life during continuous monitoring. The flow scales sampling rates based on patient activity to extend portable operation time. Low-battery warnings are automatically routed to nursing stations for timely intervention.

FIG. 5: CLINICAL VALIDATION PROCESS
FIG. 5A: DATA COLLECTION FROM PATIENTS—Outlines Data collection process from patients in hospital settings via TEE security. The process follows a structured protocol to ensure high-quality data for model training. Patient consent and data anonymization steps are integrated into the collection flow.
FIG. 5B: CLINICAL TRIAL PROTOCOL—Depicts FDA-approved protocol ensuring 95% sensitivity and 98% specificity. The trial design compares system alerts against clinical diagnosis by board-certified neurologists. Milestones for statistical significance are detailed for each phase of the trial.

FIG. 5C: ACCURACY METRIC EVALUATION—Shows evaluation of accuracy metrics derived from clinical validation data. Evaluation includes the calculation of true positive and false positive rates across diverse patient groups. Results are used to validate the final risk classifier before deployment.
FIG. 5D: FEEDBACK LOOP IMPLEMENTATION—Illustrates loop for model refinement using real-time clinical data. The loop allows the system to learn from clinician-verified outcomes to reduce future errors. Updates are deployed securely to the TEE through authenticated hospital network channels.
FIG. 5E: REGULATORY COMPLIANCE CHECK—Describes process for ensuring compliance with FDA 510(k) and ISO standards. The check includes rigorous testing of system security and clinical efficacy. Documentation is generated automatically to support regulatory submissions.

### DETAILED DESCRIPTION OF THE INVENTION

The predicate diagnostic system for detecting stroke and TIAs in hospital settings utilizes TEE-secured devices to ensure clinical-grade security. The sensor data aggregator employs optimized sampling for hospital patient profiles. To maintain 95% sensitivity with an optimized local dataset, the analysis engine utilizes an AI architecture initialized via transfer learning. Pre-trained weights from general neurological data are fine-tuned using hospital-specific patterns augmented by generative data augmentation (synthetic samples). This ensures the hybrid CNN-decision tree model identifies physiological deviations within high-density clinical environments while maintaining HIPAA-compliant confidentiality via AES-256 and SHA-3 hashing.

### DEFINITIONS

Adaptive Gait Baseline: A dynamic mathematical model adjusting to walking patterns in a clinical ward to distinguish pre-existing deficits from acute onset.
Cryptographic Software: A TEE-integrated module using AES-256 and SHA-3 hashing to ensure data accuracy.
Data Encryption Module: A hardware component securing multimodal data via integrated cryptographic software.
Environmental Data Filter: an algorithmic module cross-referencing movement data to suppress false alerts from clinical equipment.
Generative Data Augmentation: A process using synthetic samples to expand the diagnostic dataset, ensuring detection of rare markers despite limited physical samples.
Hospital Connectivity Protocol: A communication system linking the mobile diagnostic device to hospital networks.
Hospital Stroke Indicator: Detectable changes in movement or traits specifically processed for detection within a clinical environment.
Mobile Sensor Array: A combination of optimized sensors on TEE-secured devices supporting continuous hospital monitoring.
Predicate Rule: Conditional logic for stroke/TIA detection processed by a hybrid CNN-decision tree model.
Real-Time Alert Threshold: A predefined score triggering an immediate clinical alert in less than two seconds.
Sensor Data Aggregator: A TEE module that collects and preprocesses multimodal motion data using optimized sampling frequencies.

What is claimed is:

1. A computer-implemented system for predicate diagnostic detection of stroke and TIAs in hospital settings using TEE-secured mobile devices, comprising: a sensor data aggregator configured to collect and preprocess multimodal motion data with optimized sampling from a mobile sensor array, utilizing cryptographic software; a predicate analysis engine configured to apply predicate rules via a hybrid CNN-decision tree model and an adaptive gait baseline while cross-referencing environmental precision data; a stroke risk classifier configured to compute a cryptographically validated risk confidence score in less than one second; and an alert module configured to generate a real-time alert with less than two-second latency transmitted via a TEE-secured channel with hospital network integration.

2. A method for predicate diagnostic detection of stroke and TIAs in hospital settings using TEE-secured devices, comprising: collecting multimodal motion data and preprocessing with optimized sampling triggers; applying predicate rules using a hybrid machine learning model based on an adaptive gait baseline; computing a validated risk confidence score in less than one second; and generating an alert with a clinical location tag.

3. A non-transitory computer-readable medium storing instructions that, when executed by a processor within a TEE, cause the processor to:
collect motion data from a hospital-based array; apply predicate rules via a hybrid CNN-decision tree model and adaptive gait baseline;
compute a risk confidence score; and generate a real-time alert with hospital team routing data.

4. The system of claim 1, wherein the predicate analysis engine refines predicate rules using a machine learning model initialized via transfer learning and fine-tuned on an optimized dataset of stroke patterns utilizing generative data augmentation to maintain 95% sensitivity.

5. The system of claim 1, wherein the alert module integrates with hospital connectivity protocols to automatically log risk scores in an electronic health record.

6. The system of claim 1, wherein the sensor data aggregator utilizes specialized clinical hardware integration to ensure continuous monitoring during patient transport.

7. The system of claim 1, wherein the cryptographic software utilizes AES-256 and SHA-3 hashing.

8. The method of claim 2, further comprising implementing a feedback loop to refine the adaptive gait baseline using real-time clinician-verified data.

9. The medium of claim 3, wherein the instructions validate device synchronization using a protocol with less than 10 ms delay.

10. The system of claim 1, wherein the alert module routes alerts to specific medical teams based on integrated hospital department routing logic.
